(19) United States
(12) Patent Application Publication
(10) Pub. No.: US 2012/0323079 A1
(43) Pub. Date: Dec. 20, 2012

(54) METHOD AND APPARATUS FOR MANIPULATING THE UTERUS AND/OR CERVIX OF A PATIENT

(76) Inventors: Ayodeji Olumuyiwa Bakare, Harrisburg, PA (US); Paul DiCarlo, Middleboro, MA (US); Robert Degon, Bellingham, MA (US); Harold Martins, Newton, MA (US); Jon T. McIntyre, Newton, MA (US); Allison Waller, Milford, MA (US); Blake Sama, Boston, MA (US)

(21) Appl. No.: 13/350,525

(22) Filed: Jan. 13, 2012

Related U.S. Application Data
(60) Provisional application No. 61/432,367, filed on Jan. 13, 2011.

(51) Int. Cl. A61B 1/32; A61B 18/14; A61B 1/06

(52) U.S. Cl. 600/204

(57) ABSTRACT

Apparatus for manipulating the uterus and/or cervix of a patient, the apparatus comprising:

an elongated shaft having a proximal end and a distal end;

an annular rim connected to the distal end of the elongated shaft; and

a projection extending distally from the annular rim, the projection being coaxial with the annular rim

and having a helical thread on its outer surface.

### REFERENCE TO PENDING PRIOR PATENT APPLICATION

This patent application claims benefit of pending prior U.S. Provisional Patent Application Ser. No. 61/432,367, filed Jan. 13, 2011 by Ayodeji Olumuyiwa Bakare for A SINGLE PIECE COLPOTOMIZER DEVICE WITH A RETRACTABLE MANIPULATOR AND LED LIGHTS (Attorney's Docket No. 9654302), which patent application is hereby incorporated herein by reference.

### FIELD OF THE INVENTION

This invention relates to medical procedures and apparatus in general, and more particularly to medical procedures and apparatus for manipulating the uterus and/or cervix of a patient.

### BACKGROUND OF THE INVENTION

Various medical examinations and procedures require that the uterus and/or cervix of the patient be manipulated so that the uterus and/or cervix, and the connecting tissues attached to the uterus and/or cervix, can be properly viewed and accessed by the doctor. By way of example but not limitation, such manipulation of the uterus and/or cervix is typically required in a total laparoscopic hysterectomy, a partial laparoscopic hysterectomy, a colpotomy, etc.

To this end, prior art medical instruments have been developed for manipulating the uterus and/or cervix of a patient. These prior art medical instruments are intended to be inserted into the vagina of the patient and have (i) a first portion which contacts the cervix of the patient, and (ii) a second portion which is advanced into the uterus of the patient and is used to manipulate the intermediate and distal portions of the uterus. See FIG. 1, which shows various elements of the female anatomy, namely, a fallopian tube 5, the bladder 10, the pubic bone 15, the Gräfenberg Spot 20, the clitoris 25, the urethra 30, the vagina 35, an ovary 40, the sigmoid colon 45, the uterus 50, the fornix 55, the cervix 60, the rectum 65 and the anus 70.

Unfortunately, none of the aforementioned prior art medical instruments adequately and completely perform their function so as to facilitate rapid, easy and atraumatic manipulation of the uterus and/or cervix of a patient so as to provide adequate viewing and access by the doctor.

Thus there is a need for a new and improved method and apparatus for manipulating the uterus and/or cervix of a patient, wherein the new and improved method and apparatus facilitate rapid, easy and atraumatic manipulation of the uterus and/or cervix so as to provide adequate viewing and access by the doctor.

### SUMMARY OF THE INVENTION

These and other objects are addressed by the present invention, which comprises a new and improved method and apparatus for manipulating the uterus and/or cervix of a patient, wherein the new and improved method and apparatus facilitate rapid, easy and atraumatic manipulation of the uterus and/or cervix so as to provide adequate viewing and access by the doctor.

More particularly, in one form of the invention, there is provided apparatus for manipulating the uterus and/or cervix of a patient, the apparatus comprising:

an elongated shaft having a proximal end and a distal end;

an annular rim connected to the distal end of the elongated shaft; and
a projection extending distally from the annular rim, the projection being coaxial with the annular rim and having a helical thread on its outer surface.

In another form of the invention, there is provided apparatus for manipulating the uterus and/or cervix of a patient, the apparatus comprising:

an elongated shaft having a proximal end and a distal end;

an annular rim connected to the distal end of the elongated shaft;

a projection extending distally from the annular rim, the projection being coaxial with the annular rim; and

a guide telescopically mounted to the projection.

In another form of the invention, there is provided apparatus for manipulating the uterus and/or cervix of a patient, the apparatus comprising:

an elongated shaft having a proximal end and a distal end; and

an annular rim connected to the distal end of the elongated shaft;

wherein the rim comprises at least one light.

In another form of the invention, there is provided a method for manipulating the uterus and/or cervix of a patient, the method comprising:

providing apparatus comprising:
an elongated shaft having a proximal end and a distal end;
an annular rim connected to the distal end of the elongated shaft; and
a projection extending distally from the annular rim, the projection being coaxial with the annular rim and having a helical thread on its outer surface;

advancing the apparatus down the vagina of a patient so that the helical thread of the projection engages the cervix; and

turning the apparatus so that interaction between the helical thread and the cervix advances the apparatus so that the projection is disposed within the uterus and the annular rim engages the vaginal wall at the distal end of the fornix.

In another form of the invention, there is provided a method for manipulating the uterus and/or cervix of a patient, the method comprising:

providing apparatus comprising:
an elongated shaft having a proximal end and a distal end;
an annular rim connected to the distal end of the elongated shaft;
a projection extending distally from the annular rim, the projection being coaxial with the annular rim; and
a guide telescopically mounted to the projection;

advancing the apparatus down the vagina of a patient so that the projection engages the cervix and enters the uterus; and

telescopically advancing the guide further into the uterus.

In another form of the invention, there is provided a method for manipulating the uterus and/or cervix of a patient, the method comprising:

providing apparatus comprising:
an elongated shaft having a proximal end and a distal end; and
an annular rim connected to the distal end of the elongated shaft;
wherein the rim comprises at least one light;

advancing the apparatus down the vagina of a patient; and

using the at least one light to illuminate the anatomy of the patient.

### BRIEF DESCRIPTION OF THE DRAWINGS

These and other objects and features of the present invention will be more fully disclosed or rendered obvious by the following detailed description of the preferred embodiments of the invention, which is to be considered together with the accompanying drawings wherein like numbers refer to like elements and further wherein:

FIG. 1 is a schematic view showing various aspects of the female anatomy;

FIGS. 2-17 are schematic views showing a new and improved instrument for manipulating the uterus and/or cervix of a patient;

FIG. 18 is a schematic view of another cup which may be used with the instrument of FIGS. 2-17;

FIGS. 19-21 are schematic views of still another cup which may be used with the instrument of FIGS. 2-17;

FIG. 22 is a schematic view of yet another cup which may be used with the instrument of FIGS. 2-17;

FIGS. 23-26 are schematic views of another cup which may be used with the instrument of FIGS. 2-17; and

FIGS. 27 and 28 are schematic views of still another cup which may be used with the instrument of FIGS. 2-17.

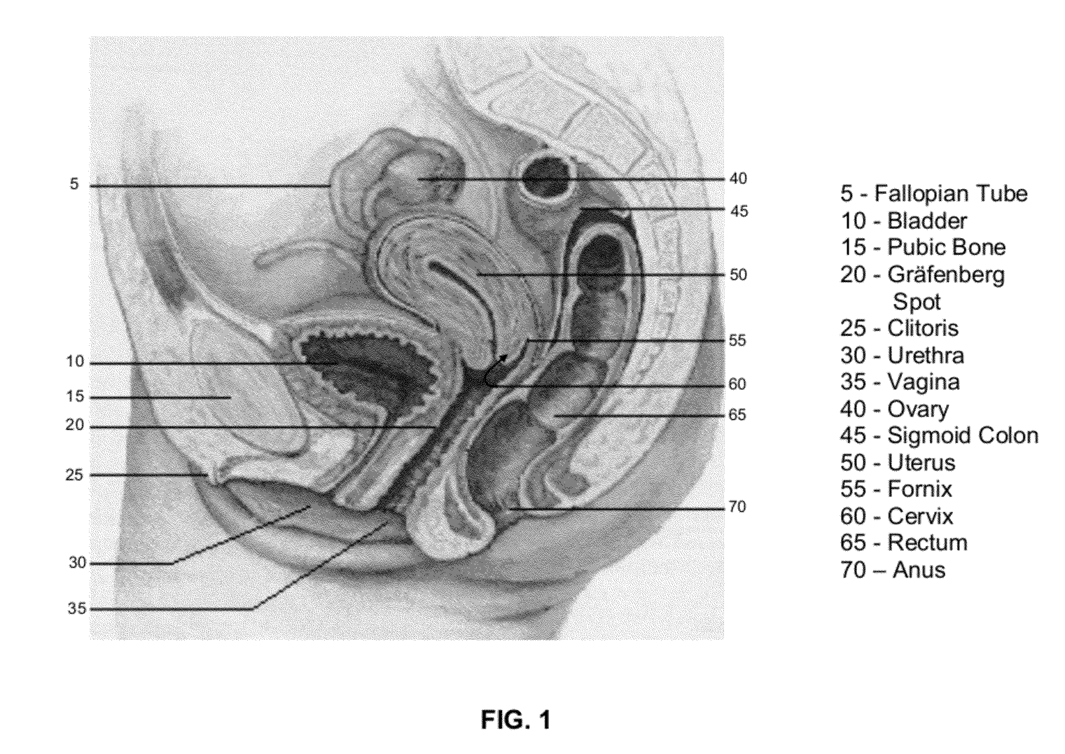

### DETAILED DESCRIPTION OF THE PREFERRED EMBODIMENTS

The present invention comprises an improved method and apparatus for manipulating the uterus and/or cervix of a patient, wherein the new and improved method and apparatus facilitate rapid, easy and atraumatic manipulation of the uterus and/or cervix so as to provide adequate viewing and access by the doctor.

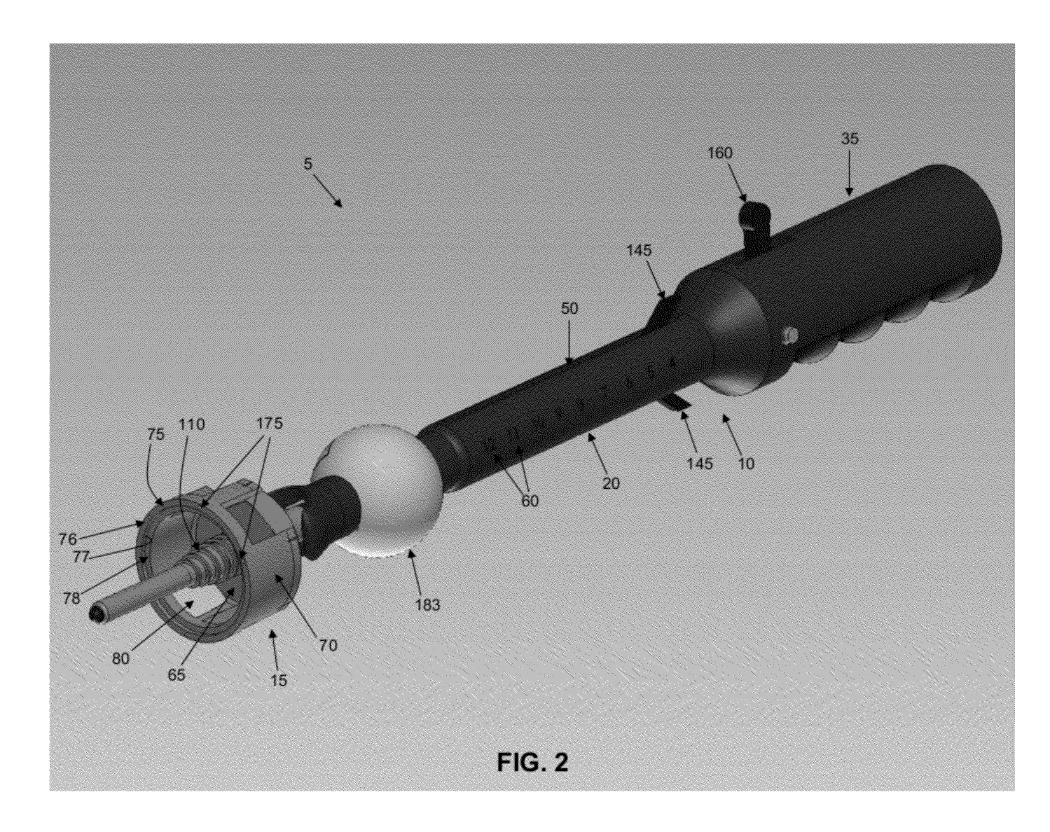

More particularly, in one form of the invention, and looking now at FIGS. 2-5, there is shown a novel instrument 5 for manipulating the uterus and/or cervix of a patient. Instrument 5 generally comprises a hollow housing 10 and a cup 15.

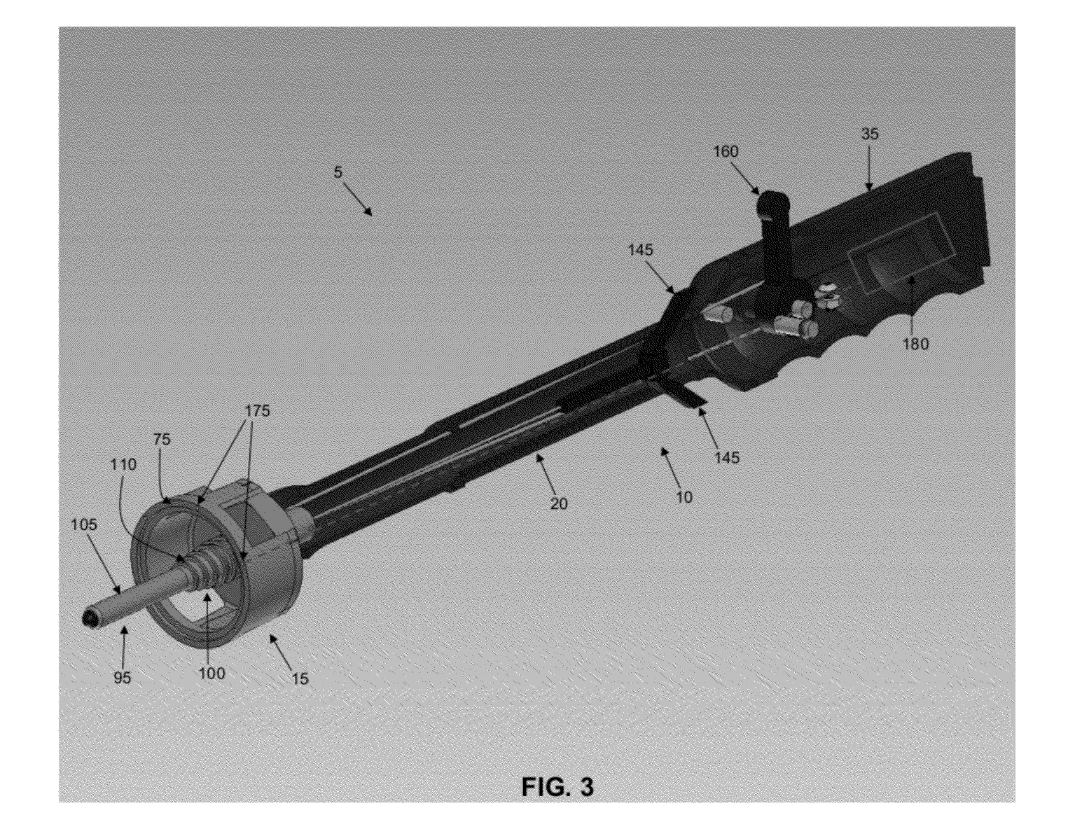

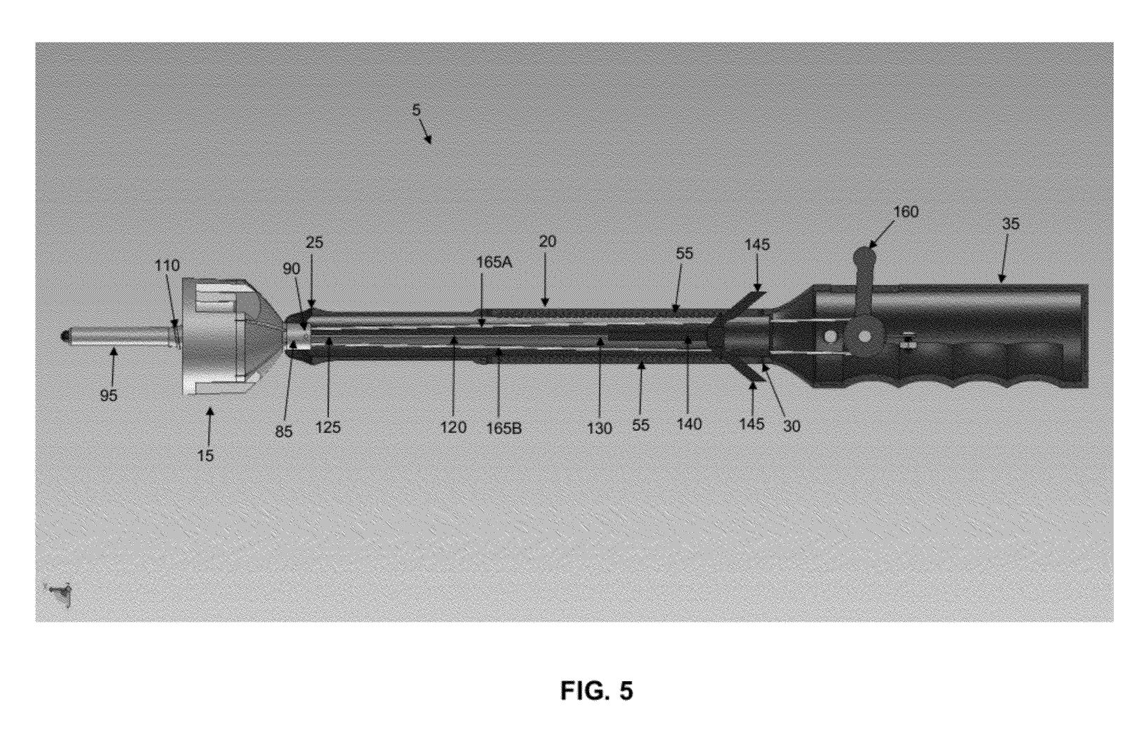

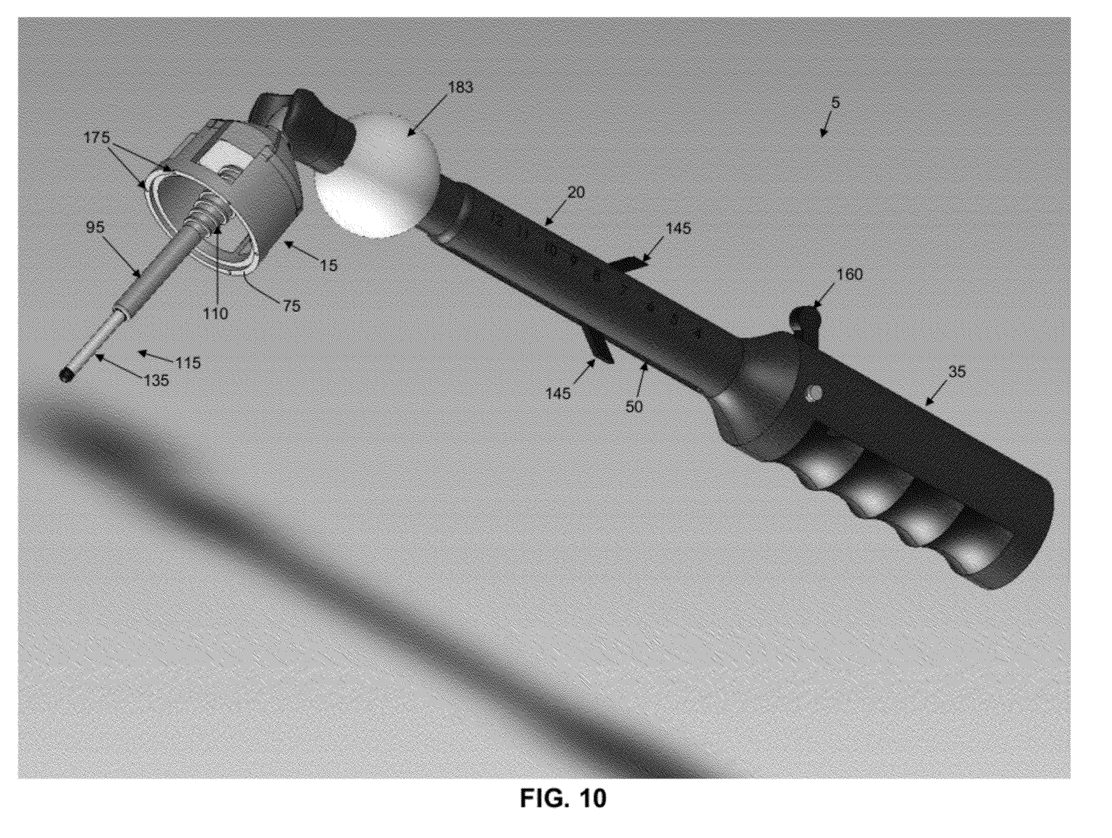

Hollow housing 10 comprises a hollow shaft 20 having a distal end 25 and a proximal end 30, and a hollow handle 35 having a distal end 40 and a proximal end 45. The proximal end 30 of hollow shaft 20 is secured to the distal end 40 of hollow handle 35 so as to form a singular hollow structure. See FIGS. 3 and 5. A pair of diametrically-opposed slots 50 (FIGS. 2 and 10) extend through the sidewall of hollow shaft 20. Ratchet teeth 55 are disposed adjacent to the inner surfaces of the diametrically-opposed slots 50. Length markings 60 are preferably located on the outside surfaces of hollow shaft 20, between diametrically-opposed slots 50.

Cup 15 comprises a base 65 (FIG. 2). An annular sidewall 70 extends distally from base 65 and terminates in a distal rim 75. Preferably distal rim 75 comprises a stepped configuration consisting of a distal outboard surface 76, an annular inner surface 77, and a proximal inboard surface 78. Preferably one or more windows 80 are formed in annular sidewall 70 of cup 15 so as to provide the doctor with visual access to the interior of cup 15. A hollow stem 85 (FIG. 5) extends proximally from base 65. Hollow stem 85 is pivotally mounted to the distal end 25 of hollow shaft 20, e.g., via a pivot pin 90, so that cup 15 can articulate relative to hollow shaft 20 and hence relative to hollow housing 10, as will hereinafter be discussed in further detail. The interior of hollow stem 85 communicates with the interior of hollow shaft 20 (and hence communicates with the interior of hollow housing 10).

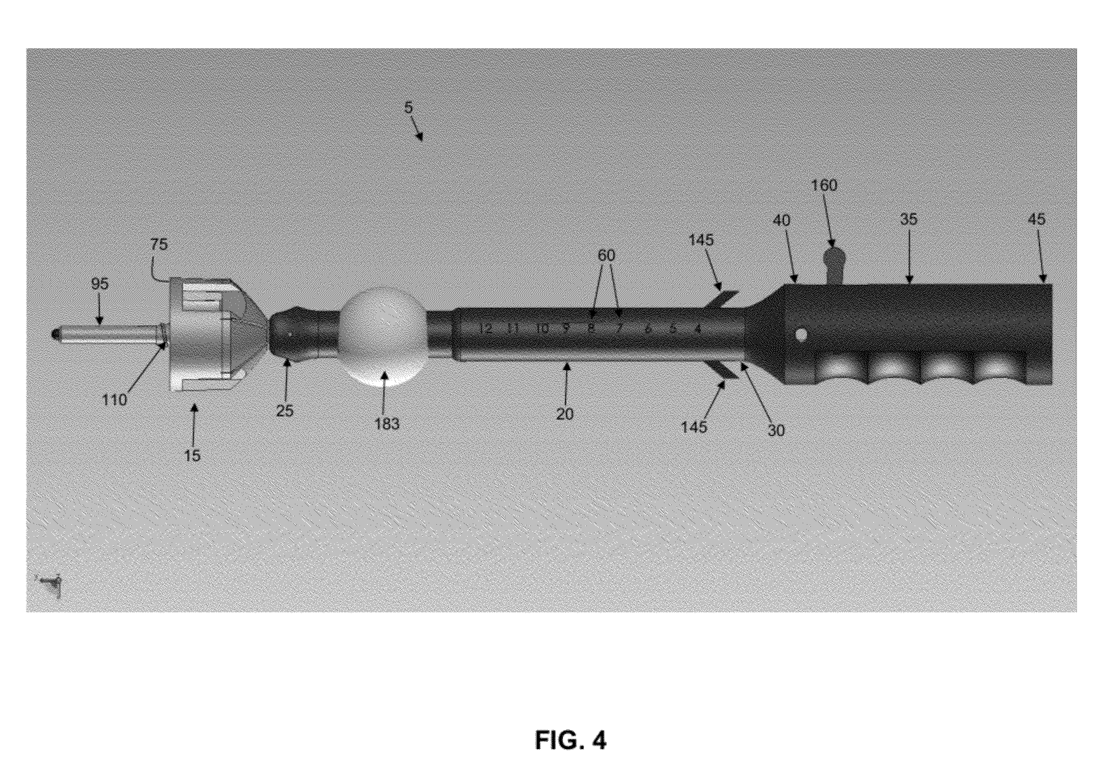

A hollow projection 95 (FIG. 3) is mounted to the distal surface of base 65 and projects distally therefrom, concentric with annular sidewall 70 of cup 15. Preferably the distal tip of hollow projection 95 extends a substantial distance beyond distal rim 75 of cup 15 (see FIG. 4). Hollow projection 95 comprises a proximal frustoconical portion 100 and a distal tubular portion 105. Proximal frustoconical portion 100 includes a helical thread 110 thereon. In one preferred form of the invention, proximal frustoconical portion 100 and helical thread 110 extend distal to distal rim 75 of cup 15 (see FIG. 4). Helical thread 110 is preferably atraumatic to tissue. An opening (not shown in the figures) in base 65 of cup 15 connects the interior of hollow projection 95 with the interior of hollow stem 85 (and hence with the interior of hollow housing 10).

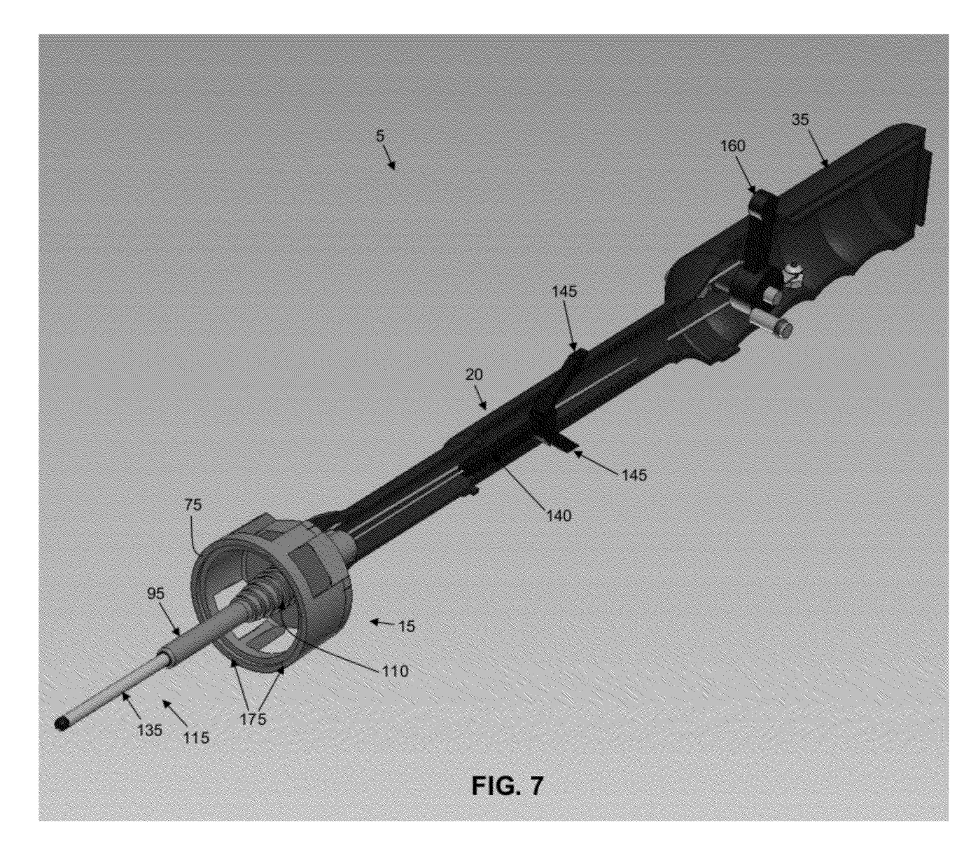

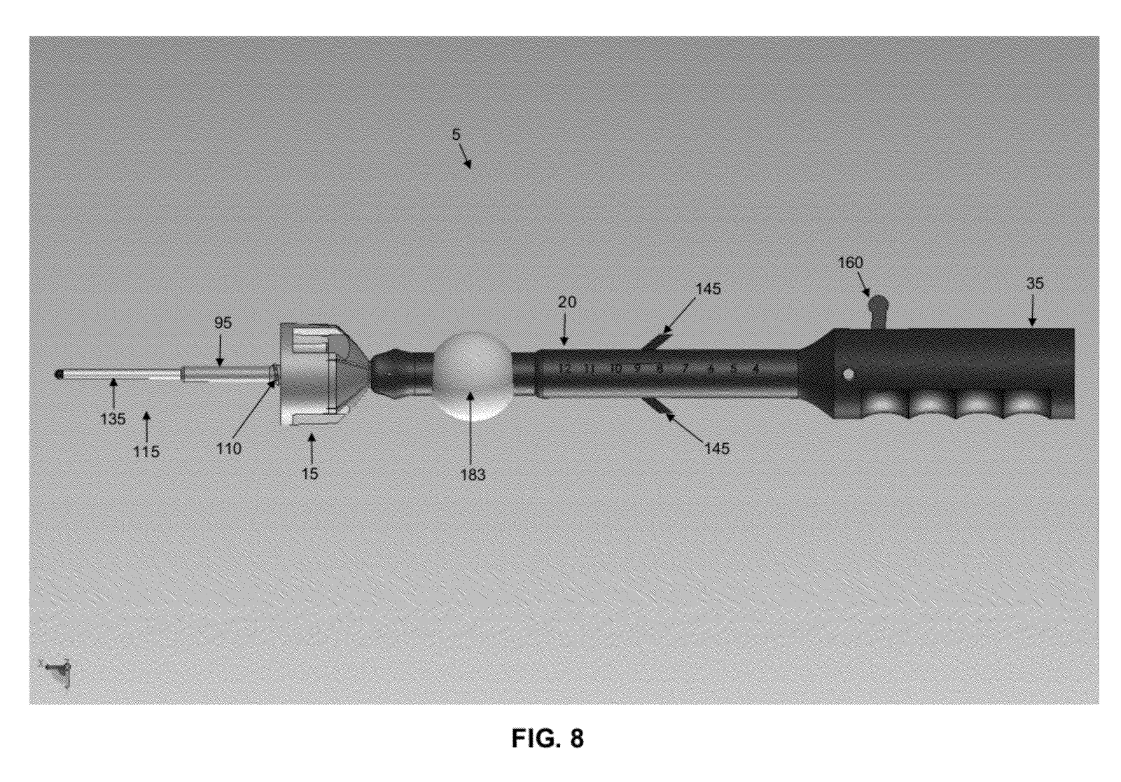

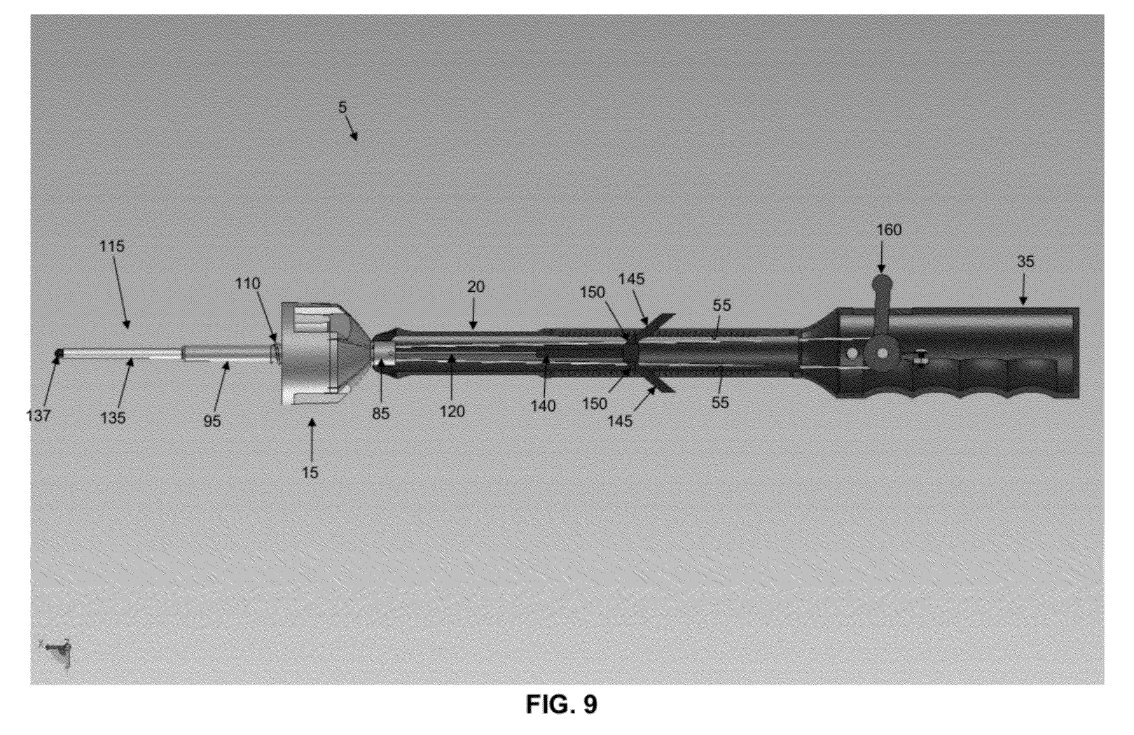

Looking now at FIGS. 2-5 and 6-9, a guide assembly 115 (FIG. 9) is movably mounted to hollow shaft 20 and cup 15. More particularly, guide assembly 115 comprises a push rod 120 (FIG. 5) having a distal end 125 and a proximal end 130. A guide 135 (FIG. 9) is mounted to the distal end 125 of push rod 120, and a pusher 140 (FIG. 5) is mounted to the proximal end 130 of push rod 120. Guide assembly 115 is movably mounted to hollow shaft 20 and cup 15, with push rod 120 extending out of hollow shaft 20, through hollow stem 85 and into hollow projection 95, with guide 135 being disposed in telescoping relation to hollow projection 95, and with pusher 140 being adjustably mounted to hollow shaft 20 (and hence adjustably mounted to hollow housing 10).

Push rod 120 is preferably formed out of a shape memory material having superelastic properties (e.g., Nitinol), such that push rod 120 can extend between hollow shaft 20 and cup 15 regardless of the angular disposition of cup 15 relative to hollow shaft 20, as will hereinafter be discussed.

Guide 135 preferably includes an atraumatic tip 137 (FIG. 9) at its distal end.

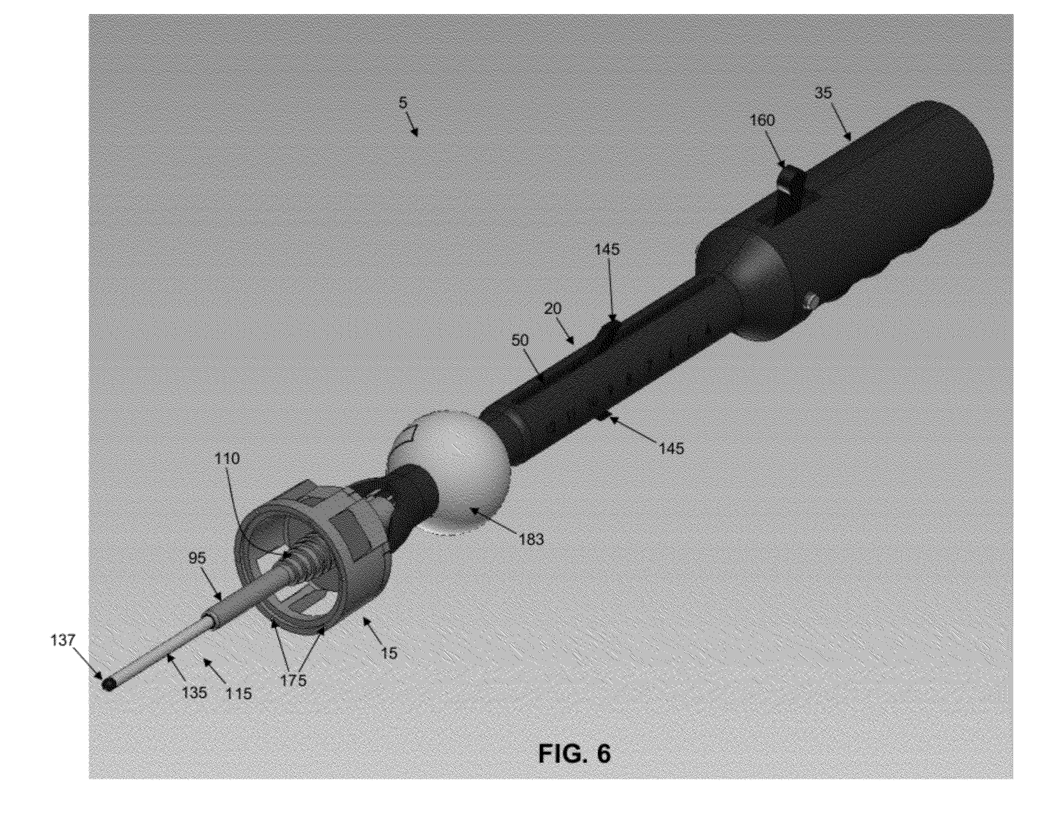

Pusher 140 comprises a pair of flexible, diametrically-opposed fingers 145 (FIG. 9) which extend radially out of hollow shaft 20 through the aforementioned diametrically-opposed slots 50. Flexible fingers 145 include edges 150. Flexible fingers 145 are biased so that edges 150 of flexible fingers 145 normally engage the aforementioned ratchet teeth 55 of hollow shaft 20, however, flexible fingers 145 may be squeezed inwardly by the doctor so as to withdraw edges 150 from ratchet teeth 55. Thus it will be seen that when flexible fingers 145 are pressed inboard, edges 150 are separated from ratchet teeth 55 and guide assembly 115 is free to move longitudinally relative to hollow housing 10 and cup 15, and when flexible fingers 145 are not pressed inboard, edges 150 engage ratchet teeth 55 and prevent longitudinal movement of guide assembly 115 relative to hollow housing 10 and cup 15. As a result of this construction, flexible fingers 145 can be used to move guide assembly 115 between (i) its retracted position (FIGS. 2-5) where atraumatic tip 137 is disposed just distal to the distal end of hollow projection 95, and (ii) its projected position (FIGS. 6-9) where atraumatic tip 137 is disposed well distal to the distal end of hollow projection 95.

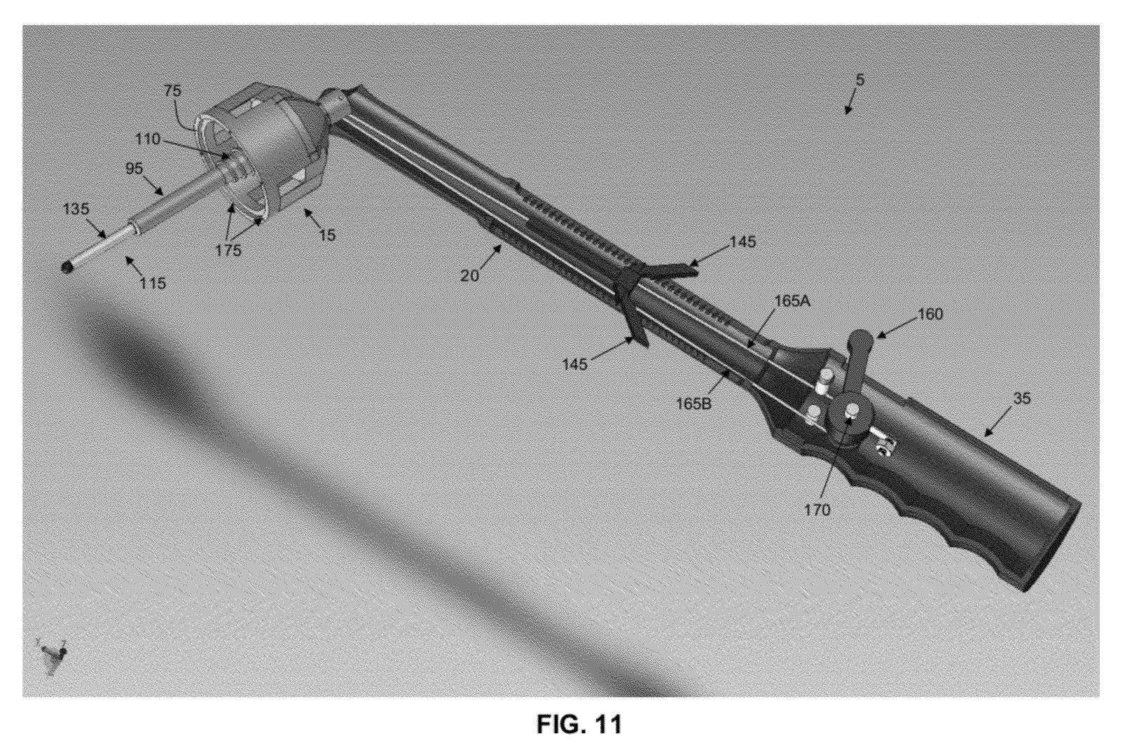

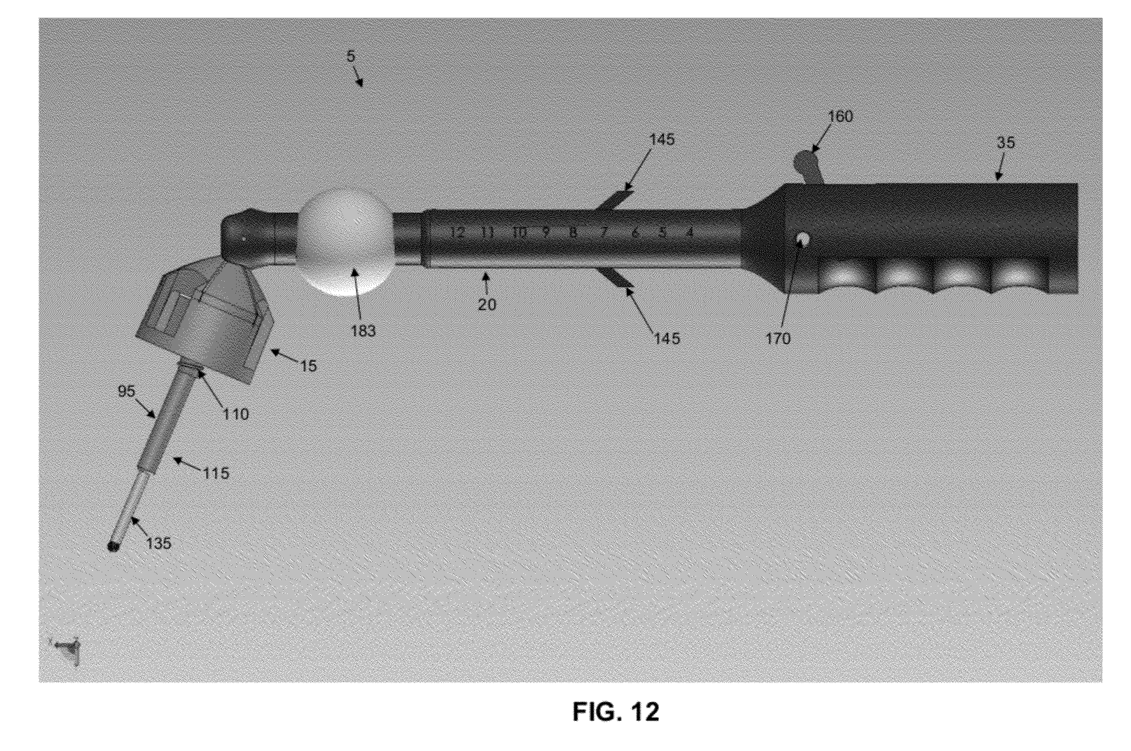

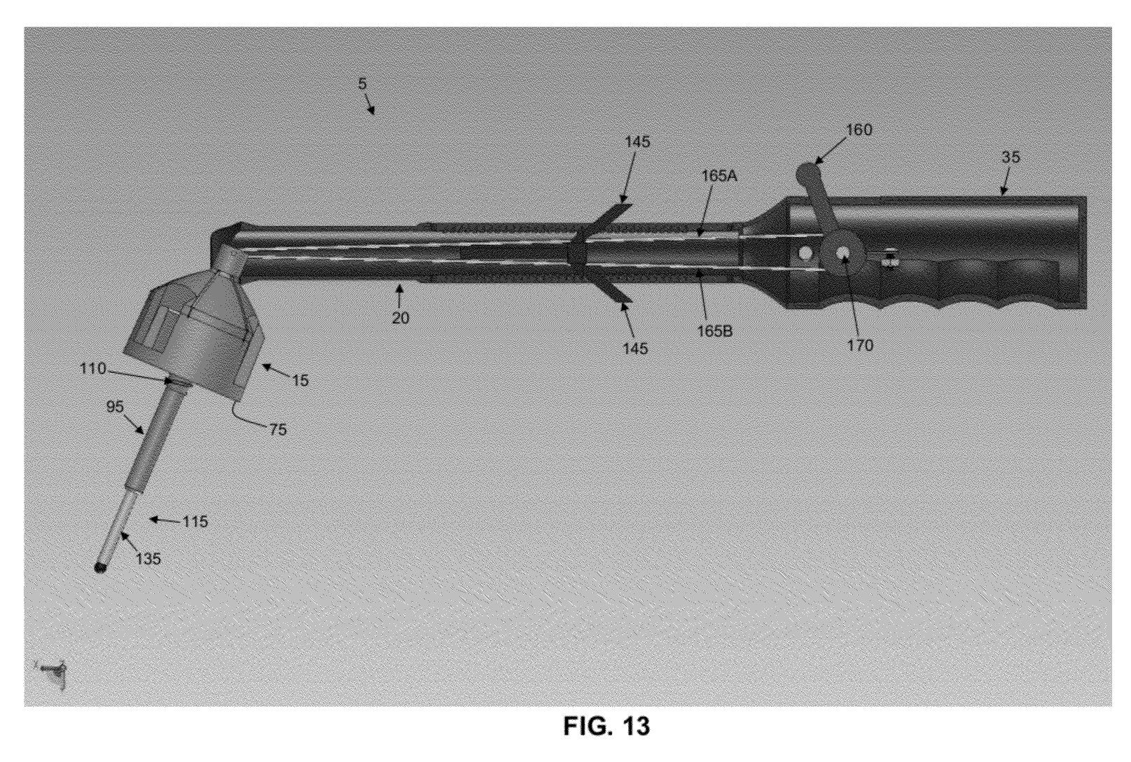

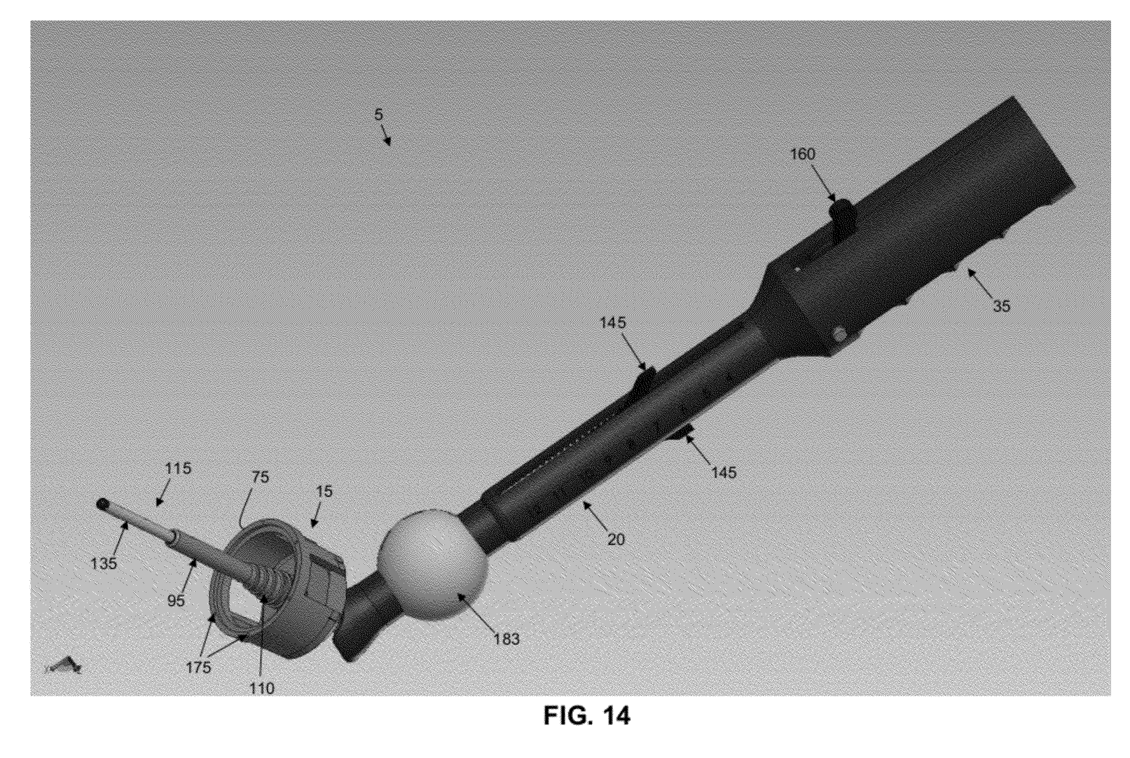

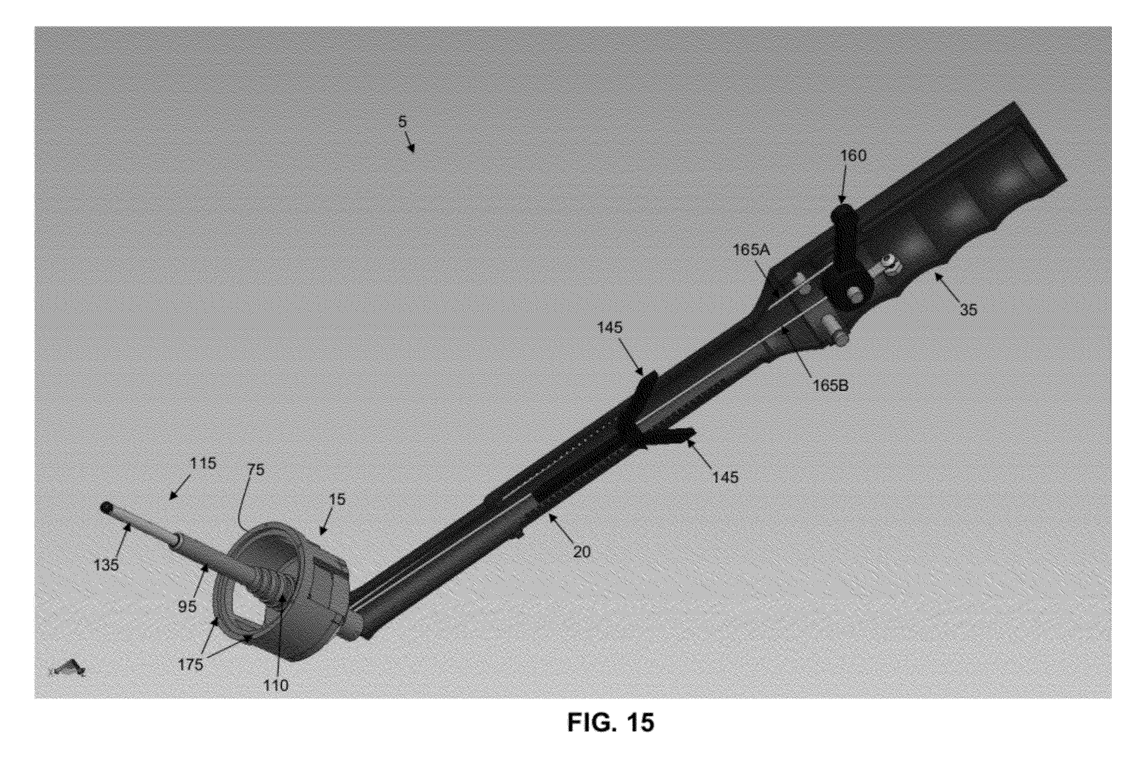

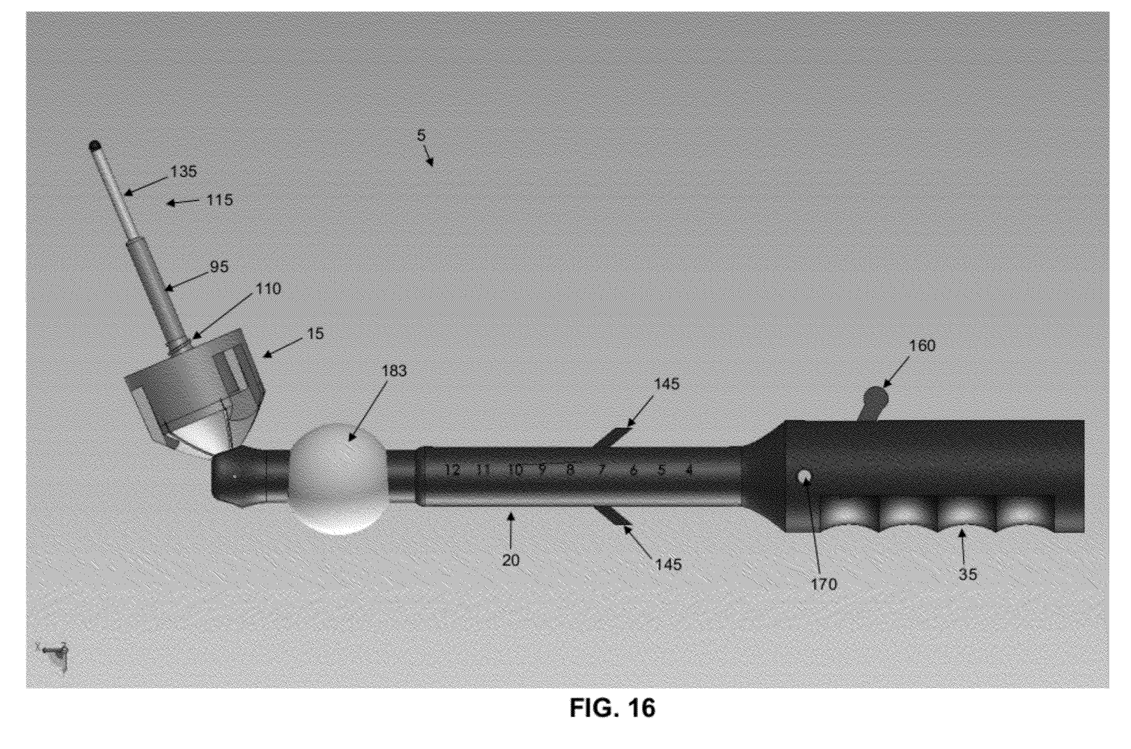

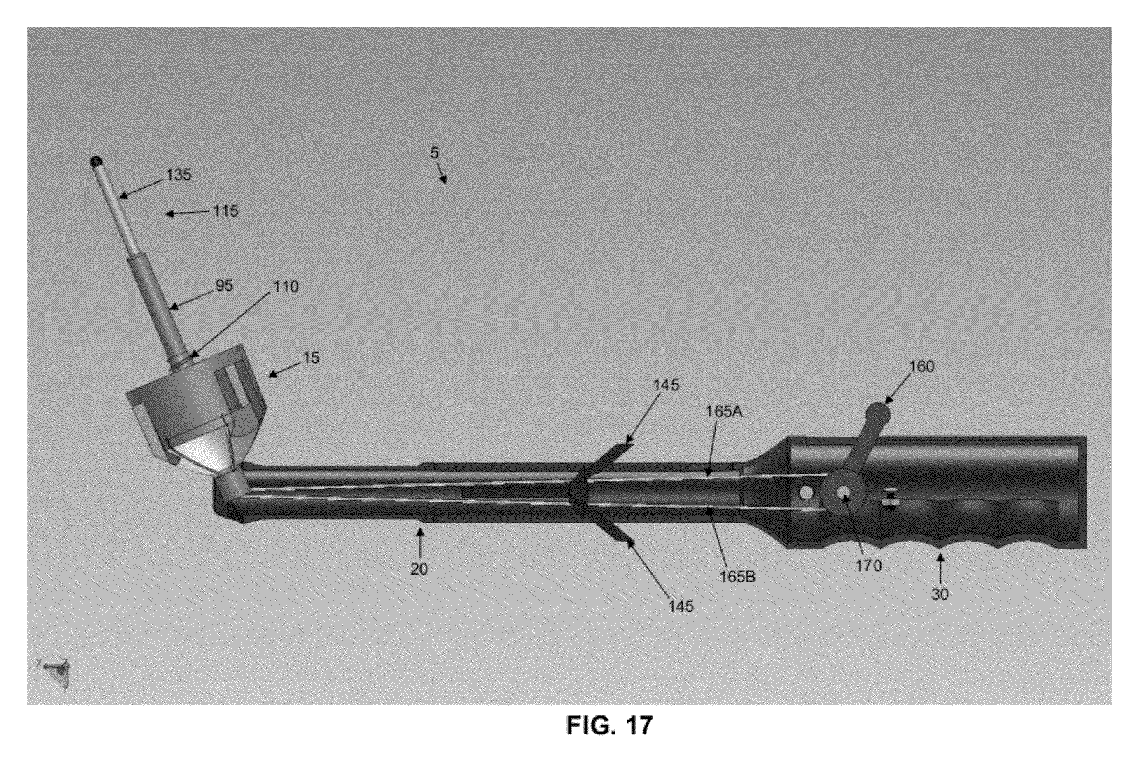

Looking next at FIGS. 10-17, a lever 160 (FIG. 11) and connecting rods 165A, 165B are provided for moving cup 15 relative to hollow housing 10. More particularly, lever 160 is pinned to handle 35 via a pivot pin 170, and connecting rods 165A, 165B connect cup 15 to lever 160, such that distal movement of lever 160 causes cup 15 to pivot downward (from the angle of view seen in FIG. 4) in the manner shown in FIGS. 10-13, and proximal movement of lever 160 causes cup 15 to pivot upward (from the angle of view seen in FIG. 4) in the manner shown in FIGS. 14-17. In order to allow cup 15 to be articulated with substantial force (e.g., to move the cervix of a patient), connecting rods 165A, 165B are preferably formed out of a relatively strong, firm material (e.g., stainless steel). At the same time, in order to allow push rod 120 of guide assembly 115 to accommodate the aforementioned articulation of cup 15 relative to hollow housing 10, push rod 120 of guide assembly 115 is preferably formed out of a superelastic material.

Preferably a light source is provided about the perimeter of distal rim 75 of cup 15. In one preferred form of the invention, a plurality of light fibers 175 (FIG. 3) are disposed about substantially the entire perimeter of distal rim 75 of cup 15 and extend from distal rim 75 of cup 15 back through the cup and hollow shaft 20 to a light source 180 contained within handle 35. In this way, light can be delivered from light source 180 to the distal end of instrument, e.g., about the perimeter of distal rim 75 of cup 15. Alternatively, LEDs can be mounted to the distal rim 75 of cup 15, with wires extending from the LEDs to a power source (not shown) contained within handle 35. As noted above, in one preferred embodiment of the present invention, light fibers 175 are sufficient in number and disposition to provide light about substantially the entire perimeter of distal rim 75 of cup 15.

A seal 183 (FIG. 2) is slidably mounted to the outer surface of hollow shaft 20.

Instrument 5 is intended to be used for manipulating the uterus and/or cervix of a patient. In one preferred method of use, instrument 5 is set so that its cup 15 is aligned with its hollow shaft 20, and guide assembly 115 is set so that it is in its aforementioned retracted position, i.e., so that its atraumatic tip 137 is disposed just distal to the distal end of hollow projection 95. Then instrument 5 is advanced up the vagina of the patient until atraumatic tip 137 of guide 135 and distal tubular portion 105 of hollow projection 95 sit at the entrance to the cervix. Then instrument 5 is gently advanced distally so that atraumatic tip 137 of guide 135 and distal tubular portion 105 of hollow projection 95 enter the cervix and start to enter the interior of the uterus.

This pushing advancement is continued until helical thread 110 on proximal frustoconical portion 100 of hollow projection 95 engages the cervix. Further forward motion is then provided by rotationally turning instrument 5, with helical thread 110 engaging the surrounding walls of the cervix and propelling instrument 5 forward. This forward motion continues, with cup 15 advancing along the fornix and enveloping the cervix, until the distal rim 75 of cup 15 comes to rest against the vaginal wall at the distal end of the fornix. This action secures the distal end of instrument 5 to the cervix, by virtue of the threaded engagement of hollow projection 95 with the surrounding surfaces of the cervix, the envelopment of the cervix by cup 15, and the seating of distal rim 75 of cup 15 on the vaginal wall at the distal end of the fornix.

Seal 183 may then be moved distally along hollow shaft 20 until it seats on the anatomy.

Next, guide assembly 115 is moved distally so that guide 135 extends a substantial distance into the uterus. This is done by squeezing flexible fingers 145 together so as to disengage edges 150 from ratchet teeth 55, pushing flexible fingers distally along hollow shaft 20, and then releasing flexible fingers 145 so that edges 150 re-engage ratchet teeth 55. In one preferred manner of use, guide assembly 115 is moved distally so that the atraumatic distal tip 137 of guide 135 sits just adjacent to the distal end of the uterus, or just short of the distal end of the uterus. This may be achieved by measuring the size of the uterus earlier in the procedure (e.g., using a so-called “uterine sound instrument”, which measures the length of the uterus), and then using the length markings 60 on hollow shaft 20 to appropriately advance flexible fingers 145 along hollow shaft 20 (and hence appropriately advance guide 135 out of hollow projection 95).

Once guide 135 has been advanced an appropriate distance out of hollow projection 95, lever 160 may be used to adjust the orientation of cup 15 relative to hollow shaft 20, whereby to also adjust the orientation of guide 135 relative to hollow shaft 20, whereby to adjust the disposition of the uterus and/or the cervix of the patient. In this respect it will be appreciated that inasmuch as hollow projection 95 threadingly engages the surrounding walls of the cervix, cup 15 encompasses the cervix and the distal rim 75 of cup 15 seats on the vaginal wall at the distal end of the fornix, articulation of cup 15 relative to hollow shaft 20 will cause the disposition of the cervix to be adjusted, and inasmuch as guide 135 extends a substantial distance into the uterus, adjustment of the orientation of guide 135 relative to hollow shaft 20 will cause the disposition of the intermediate and distal portions of the uterus to be adjusted as well.

Significantly, the provision of a light source (e.g., light fibers 175) on distal rim 75 of cup 15 facilitates procedures in several ways. By way of example but not limitation, during the instrument's approach to the cervix, light fibers 175 provide excellent illumination of the vaginal wall, the cervix and the fornix as the instrument is advanced. By way of further example but not limitation, where instrument 5 is to be used for a total laparoscopic hysterectomy, which requires that the uterus be separated from the vaginal wall at the base of the fornix, once the distal rim 75 of cup 15 has settled against the vaginal wall at the distal end of the fornix, the light from light fibers 175 can be seen through the vaginal wall at the base of the fornix (e.g., by an endoscope positioned on the far side of the vaginal wall), thereby providing the doctor with a visual indication of the location of the fornix, and hence a visual cutting guide for severing the vaginal wall at the base of the fornix. In this respect it will be appreciated that inasmuch as light fibers 175 are preferably disposed about substantially the entire perimeter of distal rim 75 of cup 15, the instrument forms a substantially complete ring of light around the cervix at the base of the fornix so as to guide the doctor. In addition, the stepped configuration of distal rim 75 of cup 15 (i.e., the stepped configuration of distal outboard surface 76, annular inner surface 77, and proximal inboard surface 78) can provide the doctor with a tactile cutting guide to facilitate severing the vaginal wall at the distal end of the fornix.

In connection with the foregoing, it should also be appreciated that lever 160 can be used to adjust the disposition of the cervix without advancing guide 135 out of hollow projection 95, or before advancing guide 135 out of hollow projection 95. Thus, in this mode of use, cup 15 and hollow projection 95 alone will provide the engagement used to move the anatomy.

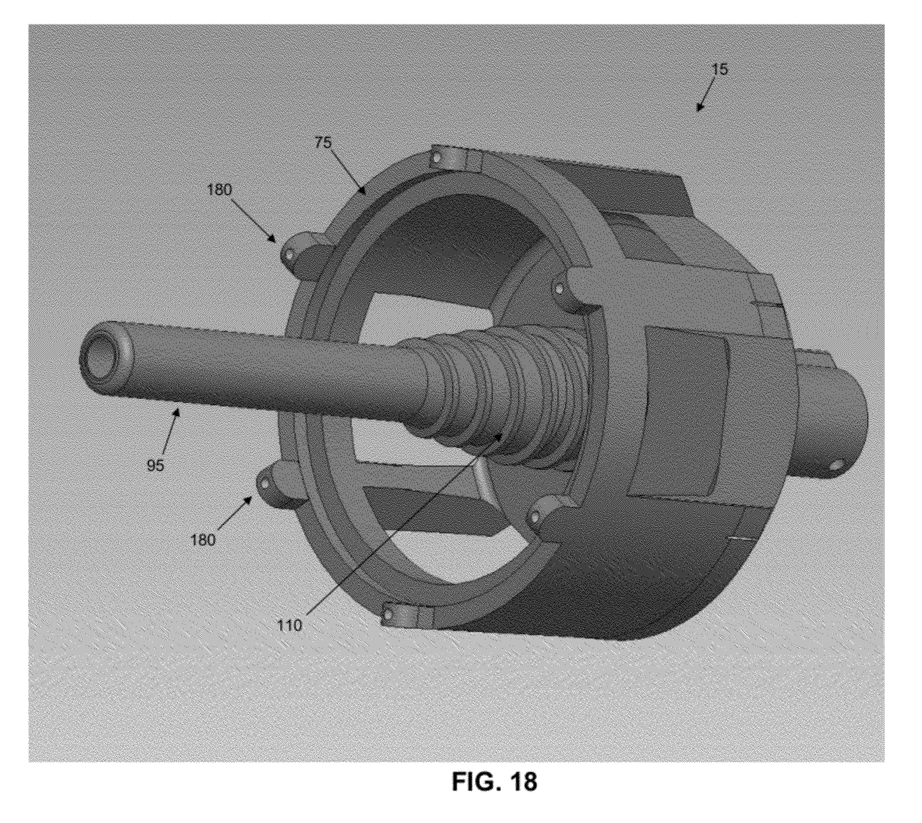

Looking next at FIG. 18, there is shown another cup 15 which may be used with instrument 5. This cup includes one or more distal projections 180. Distal projections 180 provide distal rim 75 of cup 15 with an enhanced profile, whereby to provide an enhanced tactile guide when distal rim 75 of cup 15 is seated against the vaginal wall at the distal end of the fornix.

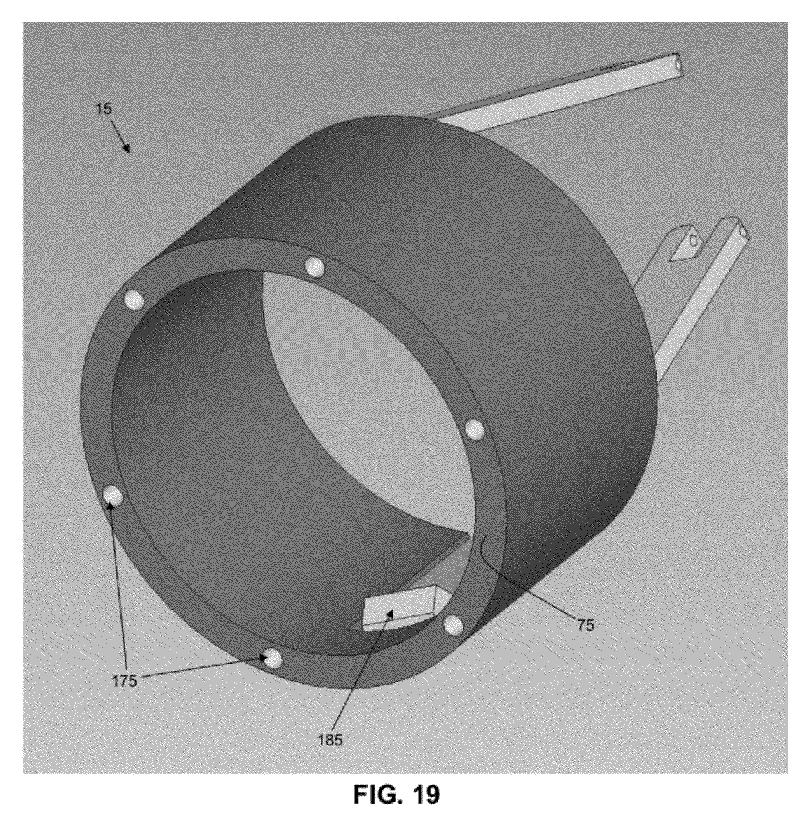

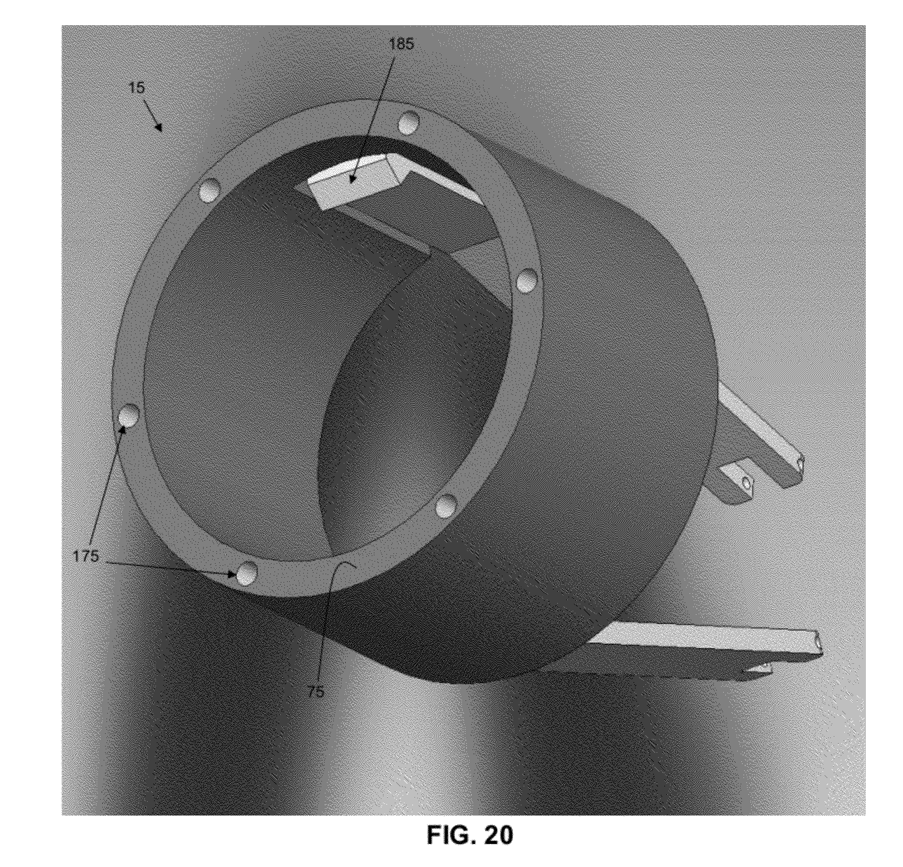

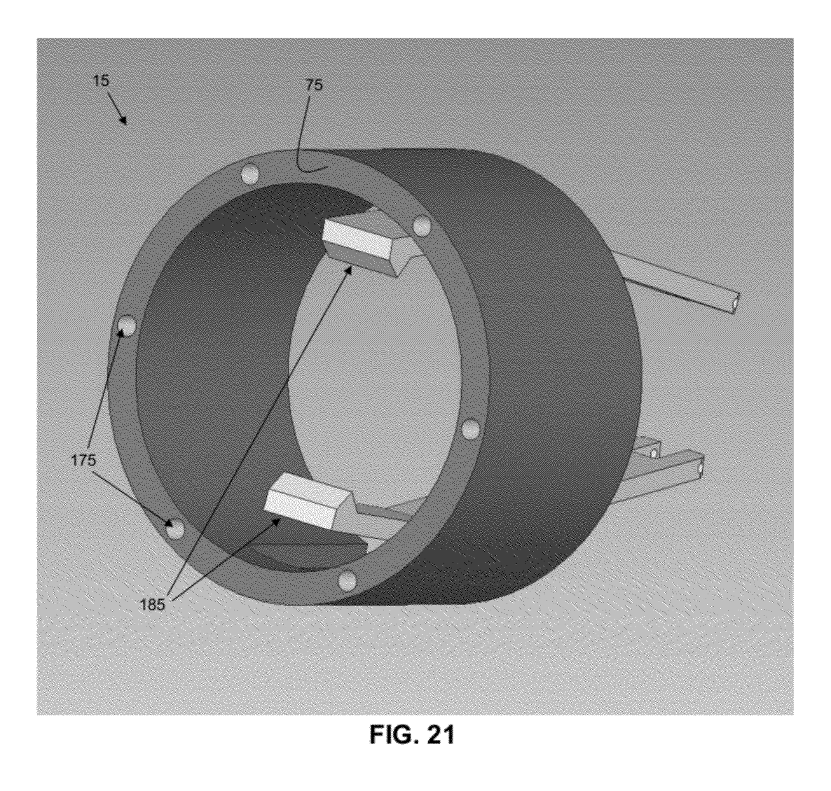

FIGS. 19-21 show another possible construction for cup 15. More particularly, in FIGS. 19-21, cup 15 includes a pair of grippers 185. Grippers 185 are arranged so as to selectively project inwardly from the interior of cup 15 so as to selectively grip the cervix of the patient, whereby to further secure instrument 5 to the anatomy of the patient.

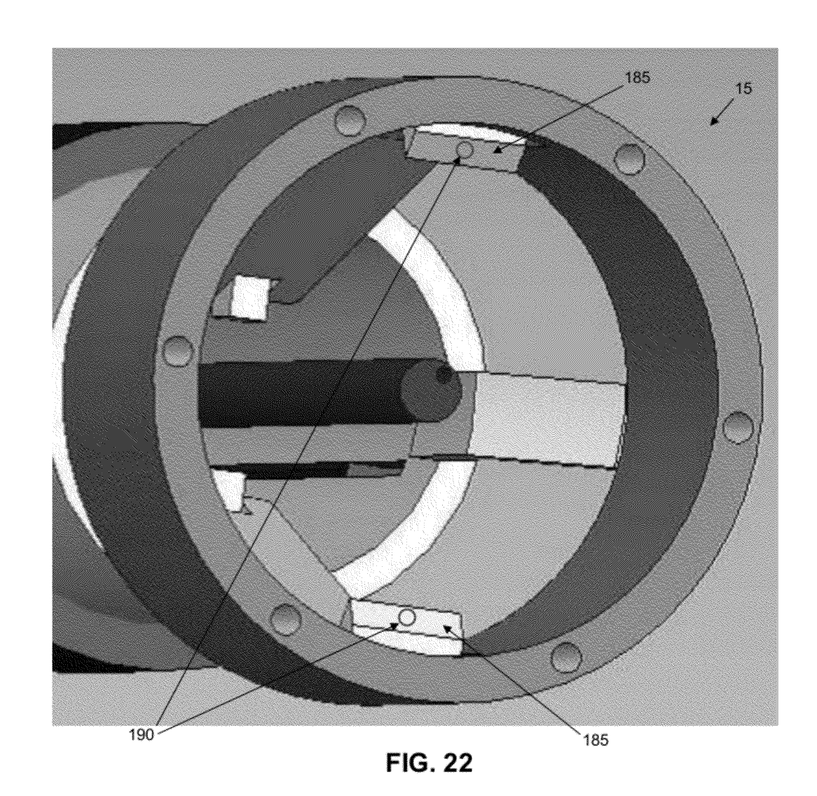

FIG. 22 shows a construction similar to that shown in FIGS. 19-21, except that light sources 190 may be provided on grippers 185. By way of example but not limitation, light sources 190 may be light fibers similar to the aforementioned light fibers 175, or light sources 190 may be LEDs, etc.

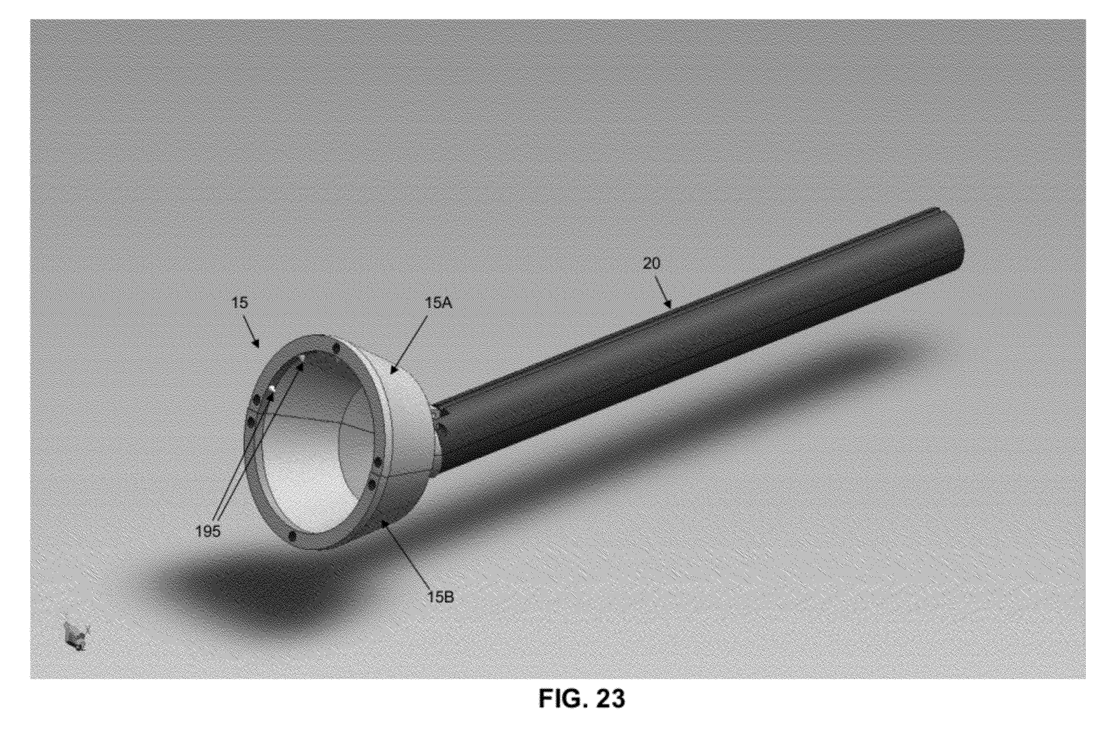

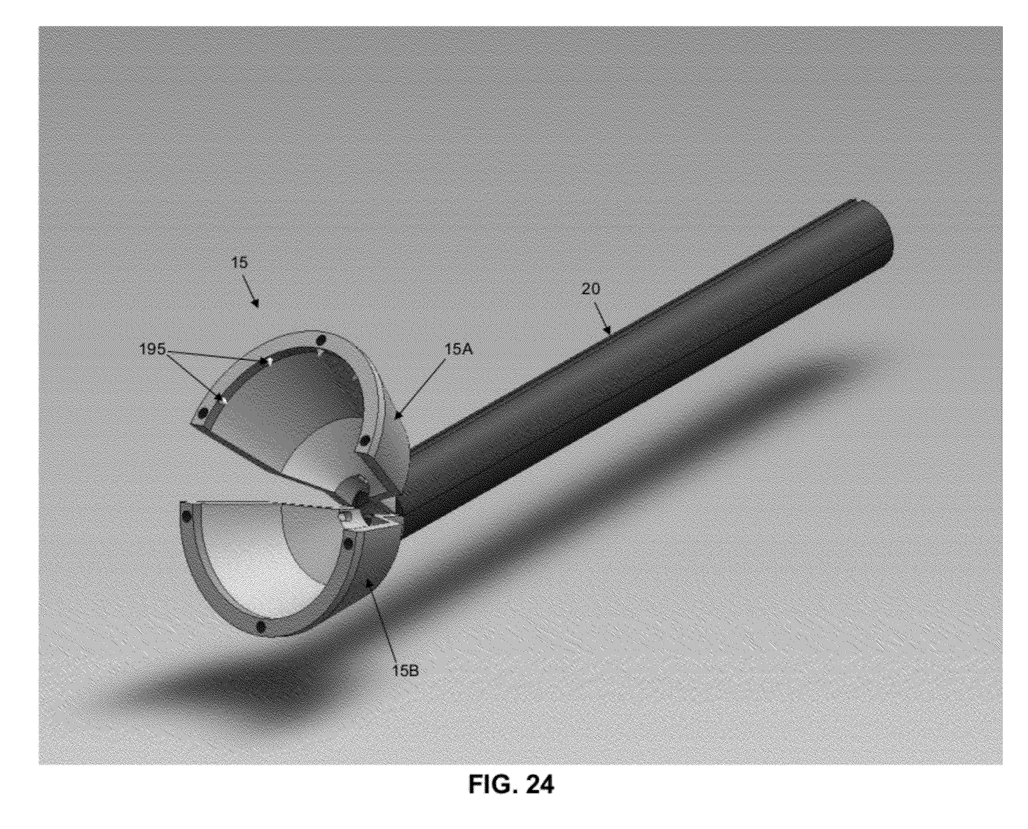

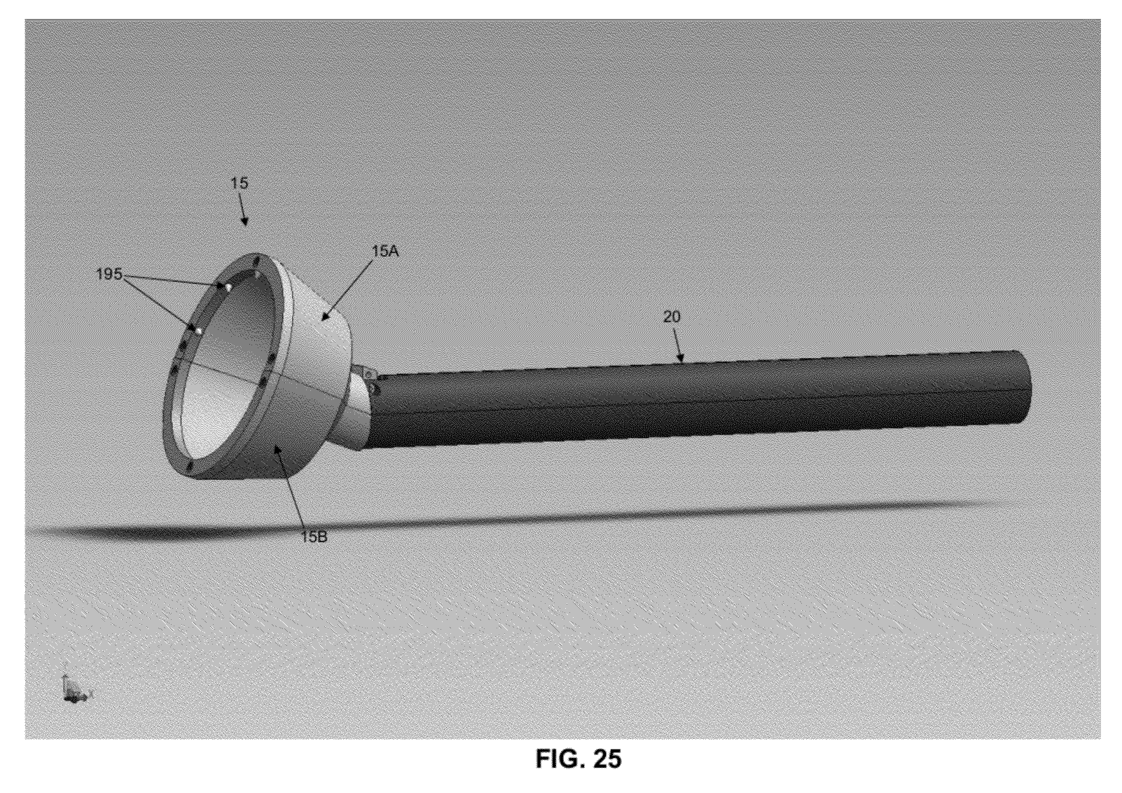

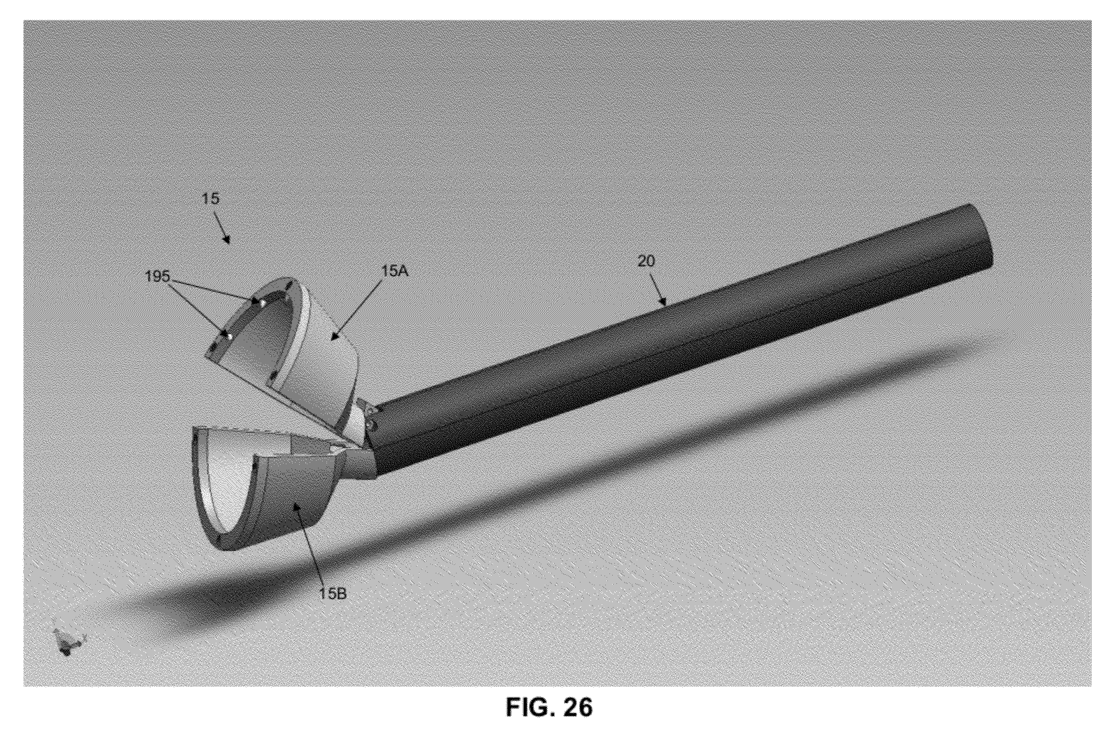

FIGS. 23-26 show still another possible construction for cup 15. In this form of the invention, cup 15 comprises two cup halves 15A, 15B, with the two cup halves 15A, 15B constructed so that they can open and close on the cervix. In this form of the invention, at least one (and preferably both) of the cup halves 15A, 15B include at least one tang 195, with tangs 195 engaging the cervix when the cup halves 15A, 15B are closed down on the cervix.

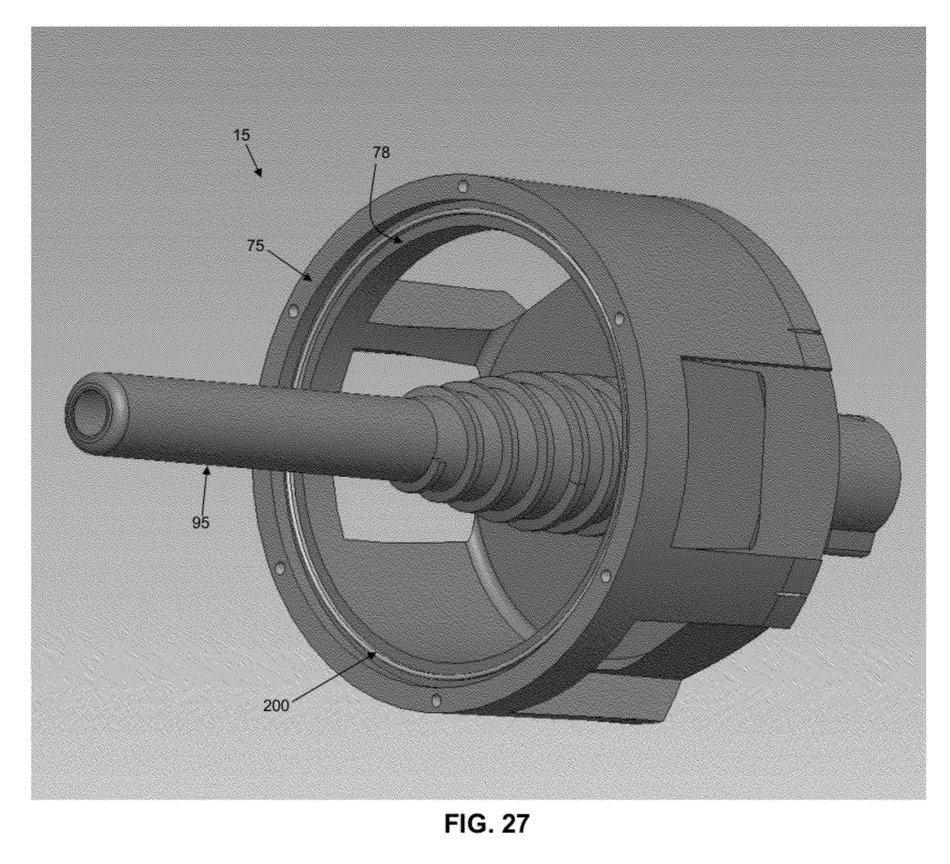

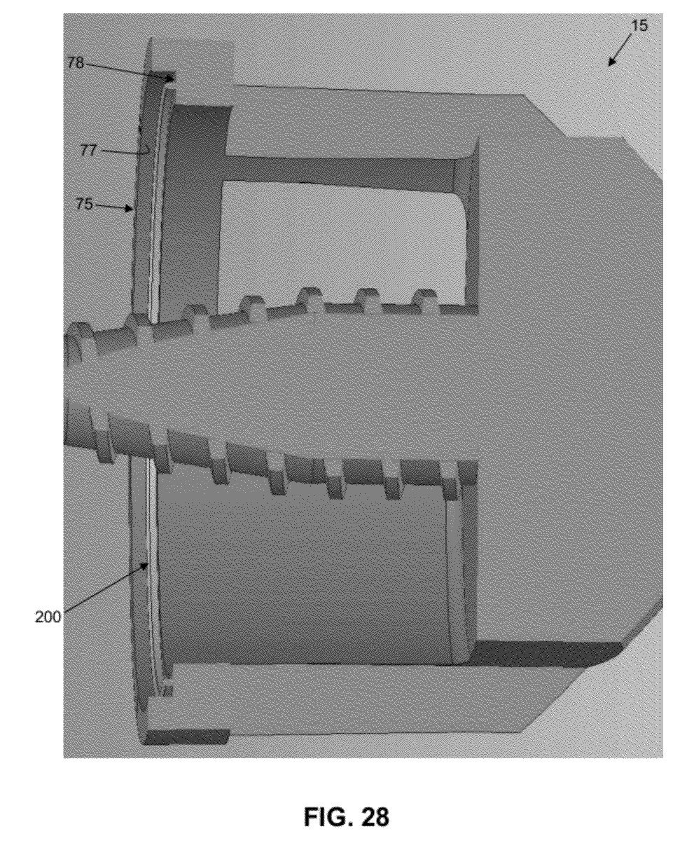

FIGS. 27 and 28 show another possible construction for cup 15. In this form of the invention, a cauterizing electrode 200 (e.g., a monopolar cauterizing electrode, a bipolar cauterizing electrode, etc.) is incorporated into the construction of the cup. Preferably cauterizing electrode 200 is disposed adjacent to proximal inboard surface 78 of the stepped distal rim 75. As a result of this construction, when instrument 5 is used in a total laparoscopic hysterectomy, where the uterus must be separated by cutting through the vaginal wall at the base of the fornix, cauterizing electrode 200 is available to cauterize the incision.

### MODIFICATIONS

While the present invention has been described in terms of certain exemplary preferred embodiments, it will be readily understood and appreciated by those skilled in the art that it is not so limited, and that many additions, deletions and modifications may be made to the preferred embodiments discussed herein without departing from the scope of the invention.

What is claimed is:

1. Apparatus for manipulating the uterus and/or cervix of a patient, the apparatus comprising:
an elongated shaft having a proximal end and a distal end;
an annular rim connected to the distal end of the elongated shaft; and
a projection extending distally from the annular rim, the projection being coaxial with the annular rim and having a helical thread on its outer surface.

2. Apparatus according to claim 1 wherein the annular rim is articulatable relative to the distal end of the elongated shaft.

3. Apparatus according to claim 1 wherein the apparatus comprises a cup, and further wherein the annular rim is formed on the distal end of the cup.

4. Apparatus according to claim 1 wherein the rim comprises at least one light.

5. Apparatus according to claim 4 wherein the rim comprises a plurality of lights which are disposed about substantially the entire perimeter of the rim.

6. Apparatus according to claim 1 further comprising a guide telescopically mounted to the projection.

7. Apparatus according to claim 1 wherein the projection comprises a frustoconical portion, and further wherein at least a portion of the helical thread is mounted to the frustoconical portion.

8. Apparatus according to claim 1 wherein the rim comprises a stepped configuration.

9. Apparatus according to claim 1 wherein the rim comprises a gripper mechanism for gripping the cervix.

10. Apparatus according to claim 1 wherein the rim comprises at least two portions, and further wherein the at least two portions are articulatable relative to one another.

11. Apparatus according to claim 1 further comprising a cauterizing electrode mounted to the annular rim.

12. Apparatus for manipulating the uterus and/or cervix of a patient, the apparatus comprising:
an elongated shaft having a proximal end and a distal end;
an annular rim connected to the distal end of the elongated shaft;
a projection extending distally from the annular rim, the projection being coaxial with the annular rim; and
a guide telescopically mounted to the projection.

13. Apparatus according to claim 12 wherein the annular rim is articulatable relative to the distal end of the elongated shaft.

14. Apparatus according to claim 12 wherein the projection has a helical thread on its outer surface.

15. Apparatus according to claim 12 wherein the apparatus comprises a cup, and further wherein the annular rim is formed on the distal end of the cup.

16. Apparatus according to claim 12 wherein the rim comprises at least one light.

17. Apparatus according to claim 16 wherein the rim comprises a plurality of lights which are disposed about substantially the entire perimeter of the rim.

18. Apparatus according to claim 12 wherein the projection comprises a frustoconical portion, and further wherein at least a portion of the helical thread is mounted to the frustoconical portion.

19. Apparatus for manipulating the uterus and/or cervix of a patient, the apparatus comprising:
an elongated shaft having a proximal end and a distal end; and
an annular rim connected to the distal end of the elongated shaft;
wherein the rim comprises at least one light.

20. Apparatus according to claim 19 wherein the rim comprises a plurality of lights which are disposed about substantially the entire perimeter of the rim.

21. Apparatus according to claim 19 further comprising a projection extending distally from the annular rim, the projection being coaxial with the annular rim and having a helical thread on its outer surface.

22. Apparatus according to claim 19 wherein the annular rim is articulatable relative to the distal end of the elongated shaft.

23. Apparatus according to claim 19 wherein the apparatus comprises a cup, and further wherein the annular rim is formed on the distal end of the cup.

24. Apparatus according to claim 19 further comprising a guide telescopically mounted to the projection.

25. Apparatus according to claim 19 wherein the projection comprises a frustoconical portion, and further wherein at least a portion of the helical thread is mounted to the frustoconical portion.

26. Apparatus according to claim 19 wherein the rim comprises a stepped configuration.

27. Apparatus according to claim 19 wherein the rim comprises a gripper mechanism for gripping the cervix.

28. Apparatus according to claim 19 wherein the rim comprises at least two portions, and further wherein the at least two portions are articulatable relative to one another.

29. A method for manipulating the uterus and/or cervix of a patient, the method comprising:
providing apparatus comprising:
an elongated shaft having a proximal end and a distal end;
an annular rim connected to the distal end of the elongated shaft; and
a projection extending distally from the annular rim, the projection being coaxial with the annular rim and having a helical thread on its outer surface;
advancing the apparatus down the vagina of a patient so that the helical thread of the projection engages the cervix; and
turning the apparatus so that interaction between the helical thread and the cervix advances the apparatus so that the projection is disposed within the uterus and the annular rim engages the vaginal wall at the distal end of the fornix.

30. A method according to claim 29 wherein the apparatus further comprises a guide telescopically mounted to the projection, and further wherein the method comprises the additional step of telescopically advancing the guide out of the projection and further into the uterus.

31. A method according to claim 30 wherein the annular rim is articulatable relative to the distal end of the elongated shaft, and further wherein the method comprises the additional step of articulating the annular rim relative to the distal end of the elongated shaft.

32. A method according to claim 29 wherein the apparatus comprises a cup, and further wherein the annular rim is formed on the distal end of the cup, and further wherein the cup envelops the cervix when the rim of the cup engages the vaginal wall at the distal end of the fornix.

33. A method according to claim 30 wherein the rim comprises at least one light, and further wherein the light is directed toward the cervix as the helical thread of the projection engages the cervix.

34. A method according to claim 29 wherein the rim comprises at least one light, and wherein the method comprises the further step of forming an incision in the vaginal wall at the distal end of the fornix using the at least one light as a light guide.

35. A method according to claim 34 wherein the rim comprises a plurality of lights which are disposed about substantially the entire perimeter of the rim.

36. A method according to claim 29 wherein the rim comprises a stepped configuration, and wherein the method comprises the further step of forming an incision in the vaginal wall at the distal end of the fornix using the stepped configuration of the rim as a tactile cutting guide.

37. A method for manipulating the uterus and/or cervix of a patient, the method comprising:
providing apparatus comprising:
an elongated shaft having a proximal end and a distal end;
an annular rim connected to the distal end of the elongated shaft;
a projection extending distally from the annular rim, the projection being coaxial with the annular rim; and
a guide telescopically mounted to the projection;
advancing the apparatus down the vagina of a patient so that the projection engages the cervix and enters the uterus; and
telescopically advancing the guide further into the uterus.

38. A method for manipulating the uterus and/or cervix of a patient, the method comprising:
providing apparatus comprising:
an elongated shaft having a proximal end and a distal end; and
an annular rim connected to the distal end of the elongated shaft;
wherein the rim comprises at least one light;
advancing the apparatus down the vagina of a patient; and
using the at least one light to illuminate the anatomy of the patient.
